(19) United States
(12) Patent Application Publication
(10) Pub. No.: US 2025/0169779 A1
(43) Pub. Date: May 29, 2025

(54) METHOD FOR DUAL-ENERGY IMAGING OF A RECORDING REGION WITH AN X-RAY FACILITY, AND X-RAY FACILITY

(71) Applicant: Siemens Healthineers AG, Forchheim (DE)

(72) Inventors: Alois Regensburger, Poxdorf (DE); Philipp Roser, Erlangen (DE)

(21) Appl. No.: 18/950,855

(22) Filed: Nov. 18, 2024

(30) Foreign Application Priority Data
Nov. 29, 2023 (DE) 10 2023 211 963.3

(51) Int. Cl. A61B 6/00; G06T 7/11

(52) U.S. Cl. CPC A61B 6/482; G06T 7/11; G06T 2207/10116

(57) ABSTRACT

Dual-energy imaging of a recording region is provided by a recording arrangement with an X-ray tube assembly and an X-ray detector for receiving X-rays of an X-ray field emitted by the X-ray tube assembly in cone beam geometry. The field has a central beam, and the recording arrangement is rotated around the recording region for recording projection data of different directions of projection for two different X-ray spectra. For each X-ray spectrum, a three-dimensional image dataset of the recording region is reconstructed from the respective projection data. The projection data of the two X-ray spectra is recorded during the rotation that covers at least 360°. For each X-ray spectrum, an associated portion of the X-ray field is fixed over the rotation, and a corresponding, associated, fixed portion of the X-ray detector is used.

### RELATED APPLICATION

This application claims the benefit of DE 10 2023 211 963.3, filed on Nov. 29, 2023, which is hereby incorporated by reference in its entirety.

### FIELD

The present document relates to a, in particular computer-implemented, method for dual-energy imaging of a recording region with an X-ray facility (system) which has a recording arrangement with an X-ray tube assembly (X-ray source) and an X-ray detector for receiving X-rays of an X-ray field emitted by the X-ray tube assembly in cone beam geometry, which field has a central beam. For recording projection data of different directions of projection for two different X-ray spectra, the recording arrangement is rotated about the recording region, and, for each X-ray spectrum, a three-dimensional image dataset of the recording region is reconstructed from the respective projection data. The present document also relates to an X-ray facility.

### BACKGROUND

In X-ray imaging, for example in a medical application, it is known to reconstruct higher dimensional image datasets from lower dimensional projection images, in particular three-dimensional image datasets (for example as a stack of sectional images) from two-dimensional projection images. In this connection, a recording arrangement, by way of example, with an X-ray tube assembly and an X-ray detector, can be moved around the recording region of an object to be recorded to record the projection images from different directions of projection. The X-ray tube assembly moves on a recording trajectory in this connection, for example a circular path.

While dedicated computed tomography facilities are known in which the X-ray tube assembly and possibly the X-ray detector are moved in a gantry, it has been proposed to implement computed tomography-like recording processes using other X-ray facilities, for example X-ray facilities with a C-arm, as are often used in angiography laboratories. This type of recording is also referred to as “DynaCT”, or, since cone beam geometry is conventionally used, “Cone Beam CT” (CBCT). An overview of the dental application of cone beam CT can be found, for example, in an article by William C. Scarfe and Allan F. Farman, “What is Cone-Beam CT and How Does it Work?”, Dent Clin N Am 62 (2008), pages 707-730.

In dual-energy imaging, two different X-ray spectra are used to record X-ray images respectively associated with them. For example, a high-energy spectrum (which can be generated, for example, in the case of a higher tube voltage) and a low-energy spectrum (which can be generated, for example, in the case of a lower tube voltage) are used. Filters are also conventionally used, also in the case of different tube voltages, to provide the respective X-ray spectrum in the desired form. These filters can differ from X-ray spectrum to X-ray spectrum. Owing to different spectral absorption properties of different materials or classes of material, for example bones and soft tissue, these materials/classes of material can be separated in a joint evaluation.

Various approaches have been proposed for the combination of CBCT and dual-energy imaging. For example, techniques have been developed to quickly switch between different tube voltages and different filters. Thus, it is possible, for example, to change between the various X-ray spectra during a rotation to be able to record projection images for the two X-ray spectra. However, a solution of this kind requires a large number of changes to customary X-ray facilities, in particular C-arm X-ray facilities, therefore a great deal of technical effort.

Approaches are also known for using what are referred to as biplane X-ray facilities which have two recording arrangements, which are operated with different X-ray spectra respectively. However, solutions of this kind can only be used in biplane systems, which are less preferable for other reasons, for example the high space requirement.

A first detector-side solution provides what are known as multi-layer detectors, in the present case two-layer detectors, which measure different X-ray spectra in the different layers. However, a dose drawback occurs here in the case of recording without spectral evaluation and a new detector development is necessary.

A second detector-side solution uses photon-counting detectors. However, technically, these can be implemented only with difficulty as a flat panel detector, so a great deal of effort and high costs for use in CBCT are to be expected.

Finally, it has also already been proposed to carry out the projection data for the two X-ray spectra completely sequentially time-wise, so the recording trajectory is firstly run though with the one X-ray spectrum and projection data is recorded for the one X-ray spectrum, after which the filter and the tube voltage are slowly changed and the recording trajectory is run though once again. However, this results in a long recording duration, and there is the risk of motion artifacts even in the case of slow patient movements.

For spiral scans, it has been proposed in the case of conventional computed tomography to use what is known as a twin beam. A filtering facility is used for this purpose that divides the X-ray field into two portions of different X-ray spectra. For example, DE 10 2008 056 891 A1 discloses a computed tomography device for carrying out a spiral scan. It includes a rotatable X-ray tube assembly and an X-ray detector positioned diametrically opposed and with an associated evaluation unit. An X-ray filter is connected downstream of the X-ray tube assembly, and its position is correlated with that of the X-ray detector. With the aid of the X-ray filter, an unfiltered and a filtered radiation portion of the fan beam are generated, with the radiation portions having different X-ray spectra. To operate the computed tomography device in a dual-energy mode, the evaluation unit evaluates a measuring signal of the unfiltered radiation portion separately from a measuring signal of the filtered radiation portion.

### SUMMARY AND DETAILED DESCRIPTION

The present approach is based on the object of disclosing a possibility of dual-energy imaging that is easy to implement and time-efficient with high-quality results for cone beam CT (CBCT).

This object is achieved by a, in particular computer-implemented, method and an X-ray facility as claimed in the coordinating claims. Advantageous developments can be found in the subclaims.

In a method of the type mentioned in the introduction, it is provided that projection data of the two X-ray spectra is recorded during the rotation that covers at least 360°, wherein for each X-ray spectrum an associated portion of the X-ray field, in particular making up at least substantially half of the X-ray field, fixed over the rotation and a corresponding, associated, fixed portion of the X-ray detector is used.

It is proposed that a complete rotation of the CBCT, covering 360°, around a fixed axis of rotation with a fixed plane of rotation, i.e., with a circular path as the recording trajectory of the X-ray tube assembly, is carried out to achieve for the X-ray spectra, in particular complete, coverage of the recording region for the two X-ray spectra despite a division of the X-ray field and thus of the X-ray detector. For this purpose, it can, in particular, be provided that the X-ray detector, more precisely its detection surface, is divided along a central line running perpendicular to the plane of rotation through the incidence of the central beam into two sides, wherein the X-ray spectra are divided into the portions antisymmetrically in respect of the central line. This means that for each point associated with one of the X-ray spectra on one side of the central line there exists a point at the same distance from the central line on the other side and which is associated with the other X-ray spectrum. This takes advantage of the fact that with a rotation covering a 360° angle of projection range, each projection beam demonstrably occurs twice in cone beam geometry. A configuration of this kind ensures that each projection beam is measured for each X-ray spectrum. Consequently, there is a complete sampling for the two sets of projection data. A three-dimensional image dataset can thus then be reconstructed for each X-ray spectrum, which can be followed by a joint evaluation to obtain items of additional information on the basis of the two X-ray spectra, in particular with regard to the distribution of material.

The simplest case for producing such an antisymmetry is that the portions are defined through the central beam by halving the X-ray field perpendicular to the plane of rotation. In this case, it is therefore provided that the one side of the X-ray detector is irradiated with the one X-ray spectrum and the other side with the other X-ray spectrum. Nevertheless, as will be demonstrated, a different division can also be expedient.

During the revolution of the recording arrangement around the recording region, projection data is simultaneously recorded for the two X-ray spectra at least within a certain tolerance. In particular, projection data is recorded for the two X-ray spectra for each recording position using the respective, disjunct portions of the X-ray field and of the X-ray detector. The projection data can be recorded simultaneously or, as will be explained below, sequentially, but extremely quickly successively, with it being possible to define the recording position as being located within an angular range with a continuous movement of the recording arrangement. It should be noted in this connection that technically a rotation about the full 360° does not have to occur to produce a 360° coverage in respect of the angle of projection, for example in the case of recording positions spaced apart by an angular distance, 360° minus the angular distance, for example in the case of 2° angular distance, 358°, are sufficient.

The X-ray facility (system) is, in particular, a C-arm X-ray facility (system), therefore one which has a C-arm to which the X-ray tube assembly and the X-ray detector are attached opposite each other. The C-arm can be rotated, in particular about its central axis, to achieve the rotation that covers a 360° angle of projection range with constant axis of rotation and plane of rotation.

A dual-energy CBCT is thereby advantageously permitted with, as will be demonstrated below, manageable changes with respect to customary X-ray facilities, i.e., easily and efficiently in terms of time.

Two possible embodiments are presented in more detail below to advantageously implement the division into portions of the X-ray spectra fixed during the rotation.

In a first embodiment, it can be provided that a filtering facility (filters, filter structure, or filter system) arranged between the X-ray tube assembly and the recording region is used for the definition of the portions, which facility has a filtering structure penetrated by the X-ray field, with a first portion for providing the first X-ray spectrum and a second portion for providing the second X-ray spectrum. On the part of the X-ray tube assembly, a single focus (focal point) is used in this embodiment, with the generated X-ray field being filtered separately so the different portions for the X-ray spectra in the X-ray field are produced after passing through the filtering facility and are produced on the X-ray detector. This has the advantage that both the tube voltage of an X-ray tube of the X-ray tube assembly as well as the filtering facility can remain static throughout the entire recording process. In other words, in respect of the hardware, only the filtering facility has to be introduced into the beam path to implement this first embodiment; other modifications are not necessary.

It should be noted in this connection that configurations are also conceivable in which no, or at least almost no, filtering is carried out for one of the X-ray spectra.

In accordance with that which has been demonstrated above, it is also possible to provide that the filtering structure is divided into two sides by a central line running perpendicular to the plane of rotation and incorporating the point of passage of the central beam through the filtering structure, and it is selected and arranged in such a way that for each point providing the first X-ray spectrum on one side of the central beam there is a point providing the second X-ray spectrum at the same distance on the other side of the central beam. In other words, the filtering structure is antisymmetrically embodied in such a way that, apart from scatter effects that potentially occur and mechanical inaccuracies, all projection beams required for a complete image reconstruction are recorded with the first X-ray spectrum and with the second X-ray spectrum respectively.

In a simple implementation of this first embodiment, it is conceivable that the one side completely provides the first X-ray spectrum while the other side completely provides the second X-ray spectrum. In this case, one half of the detector or side in respect of the central line of the X-ray detector absorbs a different X-ray spectrum to the other half of the detector/side. The filtering facility can then be formed, for example, from respective half-side filters for providing the respective X-ray spectrum.

However, it can also be conceivable in expedient developments that the portions of the filtering structure include a plurality of regions, respectively, which are separated from each other by regions of the other portion. The regions can be embodied as strips running perpendicular to the plane of rotation. All strips can have the same width, with the strips particularly advantageously having widths selected to take the inverse-square law into account, however. Owing to the cone beam geometry, the width of the strips can therefore increase outwardly in accordance with the inverse-square law. This means the width of the strips can be a function of the covered detector gaps of the X-ray detector to take into account the cone beam geometry. In particular, the widths become greater with increasing distance from the central beam or the central line of the X-ray detector to take into account the inverse-square law. In general, advantages can be achieved with regard to the correction of potential movements when two or more regions are used per X-ray spectrum.

In general, it can advantageously be provided within the context of the first embodiment that the filtering facility is introduced into the beam path by an actuator before the beginning of recording of the projection data. For example, the filtering facility can be part of a filter wheel of the X-ray facility. A control facility of the X-ray facility, which can be embodied for carrying out the method, can have a control unit (controller) to actuate the actuator for pivoting the filtering apparatus into the beam path before the beginning of the recording activity.

The filtering structure can have a multi-layer structure. Specifically, it can be provided that the multi-layered filtering structure has at least one base layer overlapping the two portions. The base layer, for example a copper layer, can therefore act on the two portions and provide a generally applicable filtering. Further, in particular, portion-specific, layers modify the X-ray radiation spectrally for providing the desired X-ray spectrum. Suitable filter materials include here, for example, gold, silver, tantalum, tungsten and the like.

In a second, alternative embodiment, it can be provided that the X-ray tube assembly has two focuses, associated with different X-ray spectra, physically spaced apart by a focal distance, which focuses follow one another in the rotational plane. The focuses are operated with different tube voltages, and the partial beam fields emitted by the focuses are separated by a shading element arranged in the beam path between the focuses, in particular, in such a way that one half of the X-ray detector respectively is associated with a partial beam field for illumination. Expediently, the focuses are provided at the same distances from the (here imaginary) central beam (of the entire X-ray field) along the plane of rotation. In this second embodiment, two X-ray focuses are used, which, in particular due to different X-ray voltages (acceleration voltages), provide different emission spectra from which the X-ray spectra. Arranged between the two focuses in the beam direction is a shading element, which can also be referred to as a central collimator element, which absorbs the X-ray radiation and thus provides for a separation of the partial beam fields. The two X-ray focuses with different tube voltages are preferably generated at two different locations of the same X-ray tube, in particular via fast, sequential switching-over of the tube voltage. The advantage of a configuration of this kind is that solely a very fast switching-over of the tube voltage is necessary, which may be easily accomplished, however, while no change of the filter or the like is necessary, which is frequently the limiting factor with regard to duration. This is achieved in that the offset focuses and the shading element provide a clearly defined geometry that physically separates the partial beam fields from one another, and different portions of the beam path and also of the X-ray detector are thus permanently associated with them. In other words, further preparation measures for providing the X-ray spectrum in the corresponding portions of the X-ray field can likewise be permanently provided. For example, it can be provided that the X-ray spectra are adjusted by X-ray spectrum-specific filters permanently introduced into the beam path of the respective partial beam fields.

The focal distance is preferably kept small and can be, for example, a few millimeters. Specifically, it can be provided that the focal distance is 0.5 to 5 mm.

It can particularly advantageously be provided that the width of the shading element is smaller than the focal distance, in particular in such a way that the partial beam fields on the detector surface of the X-ray detector, in particular at its central line, are at least substantially adjacent. The central collimator element can therefore be slightly less wide than the focal distance between the X-ray focuses, so there is no, or there is only a very small, unirradiated central strip on the X-ray detector, which can be spaced apart, for example by about 1 m, from the X-ray tube assembly, in particular the focus. It should be noted in this connection that low cross-talk between the X-ray spectra in the central region of the X-ray detector is also ultimately unproblematic since solely the spectral separation in this (small) region would be prevented.

As already mentioned, the focuses are preferably generated in the same X-ray tube of the X-ray tube assembly, wherein the different tube voltages are switched between sequentially. In other words, the focuses and the tube voltages are switched in unison. Short focus distances and corresponding small shading elements may thus also be easily implemented.

In a first variant of this second embodiment using a single X-ray tube, it can be provided that the portions of the X-ray detector sequentially illuminated by the X-ray spectra are readout in a joint readout cycle. In other words, this means that the two portions, which can be understood as partial images on the X-ray detector, can be exposed very quickly sequentially, wherein the X-ray detector can then read out the projection data of the two portions in a joint readout act. Advantageously, there are consequently no additional timing or readout demands for the X-ray detector in this variant. The only component of the X-ray facility that must be able to switch quickly is in this case the X-ray tube assembly, specifically its X-ray tube, in which there is fast switching between the one focus site with the first tube voltage and the other focus site with the second tube voltage.

In an alternative, second variant, it is also conceivable, however, that the portions of the X-ray detector sequentially illuminated by the partial beam fields are successively read out, in particular using a shadow register for at least the first of the readout processes. It is therefore possible to read out the X-ray detector between the two X-ray pulses of the corresponding X-ray spectra in quick succession, for example with the quasi-instantaneous reading out of a CMOS detector in the shadow register. In this connection, a particularly preferred development provides that with the reading out of each portion, the part of the X-ray detector not covered by the respective partial beam field, including the portion of the other partial beam field, is also read out to obtain scatter radiation data which is used in a scatter radiation correction. In such a configuration, there is therefore the possibility of using the respectively unexposed portion of the X-ray detector for a scatter radiation measurement. The scatter radiation data can then be used for image corrections. For example, it can be provided that a scatter radiation image is ascertained from the scatter radiation data also for the exposed portion of the X-ray detector and is used for correction of the projection data, in particular by way of subtraction. An improved scatter radiation correction and thus a further enhanced image quality can be achieved in this way.

In general, therefore both for the first as well as for the second embodiment, a preferred development provides that for the reconstruction of the image dataset for one of the X-ray spectra respectively, projection data of the other X-ray spectrum is taken into account. Even if the projection data of the other X-ray spectrum is not directly comparable with the projection data of the one spectrum, it can still contain useful items of information for the reconstruction, for example with regard to the truncation and/or with regard to the cone beam artifacts. The projection data of the other X-ray spectrum is not directly incorporated in the reconstruction, rather items of reconstruction information are derived from it, which can be incorporated, for example, as boundary conditions, term of a target function and the like. It is possible to show that more items of reconstruction information are available than, for example, in the case of two successively executed standard CBCT scans with rotation in an angular interval of, for example, 210°. The utilization of these additional items of reconstruction information therefore results in a clear enhancement of the image quality in dual-energy CBCT. There are thus further items of reconstruction information available, in particular in the case of an iterative and/or multispectral image reconstruction.

A specific development thus provides that a first reconstruction of preliminary datasets takes place from the respective projection image data. An item of material information in respect of the recording region, in particular describing a distribution of material, is ascertained from the preliminary image datasets and the item of material information is taken into account by at least one boundary condition and/or in a target function during at least one new reconstruction from the respective projection data. Firstly, preliminary image datasets are therefore ascertained separately from the respective projection data of the X-ray spectra as a first three-dimensional reconstruction. In summary, an item of material information, as is basically known for dual-energy imaging, can be deduced from these preliminary image datasets. This applies to the three-dimensional volume of the recording region and can provide indications as to how the projection images or the result of a more exact reconstruction would have to look. It is therefore proposed that, in particular soft, boundary conditions are deduced from the items of material information and/or, that when a target function is used, it is modified to then achieve an improvement in at least one further reconstruction process and ultimately determine the image (object) dataset. In particular, artifacts of the cone beam geometry (cone beam artifacts) can be reduced in this way.

It is possible, moreover, that projection data of the two X-ray spectra is used to ascertain a, in particular spectral, truncation model, which is taken into account in the reconstruction of the two image datasets. For example, techniques can be used as were proposed in the subsequently published German patent application DE 10 2023 204 165.7. From the projection data and using estimation methods for regions outside of the primary reconstruction volume that are not fully covered, it is possible to ascertain an entire truncation model of the patient in the region relevant to the imaging. In truncated regions in individual projection images of projection data, spectral X-ray absorption properties of the individual volume elements can be assumed on the basis of a previously conducted material classification. The truncated regions of the respective projection images can be supplemented by way of virtual forward projection by the truncation model with the first and a second X-ray spectrum. Other approaches can also be applied in this context.

Apart from the method, the present approach also relates to an X-ray facility, having
a recording arrangement with an X-ray tube assembly and an X-ray detector for receiving X-rays of an X-ray field emitted by the X-ray tube assembly in cone beam geometry, which has a central beam, and
a control facility (controller or processor) which is embodied to carry out the method.

All statements in respect of the method may be transferred to the X-ray facility, and vice versa. The X-ray facility is preferably a C-arm X-ray facility, which has a C-arm on which the X-ray tube assembly and the X-ray detector are arranged opposite each other.

The control facility can have at least one processor and at least one storage means. Functional units for carrying out acts of the method can be formed by hardware and/or software. In particular, the control facility can have a recording unit (system or controller) for controlling the rotation that covers 360° of the recording arrangement and the recording of the projection data and a reconstruction unit (system or processor) for reconstruction of the image datasets.

In the case of the first embodiment, the X-ray facility also has the filtering facility. If an actuator is provided for pivoting the filtering facility, the control facility can have a control unit (controller) for actuating the actuator. In the case of the second embodiment, the shading element is provided and the recording unit is also embodied for actuating the X-ray tube assembly to generate the two focuses.

The method can be implemented on the control facility as a computer program which, when it is executed on the control facility, causes the facility to carry out the acts of the method. The computer program can be stored on a non-transitory electronically readable data carrier (medium).

It should also be noted at this point that the use, proposed here, of different portions of the X-ray field or X-ray detector, fixed during the rotation, takes place independently of a basically possible collimation of the X-ray field by a collimation facility (collimator) of the X-ray facility. A collimation facility uses, for example, lateral blades in order to adapt the dimensions of the X-ray field per se, while the present approach divides, within the X-ray field, i.e., for the region to actually be recorded, into portions for the X-ray spectra and in the process takes advantage of the fact that the necessary information is nevertheless still fully available due to the complete rotation.

### BRIEF DESCRIPTION OF THE DRAWINGS

Further advantages and details can be found in the exemplary embodiments described below, as well as on the basis of the drawings, in which:

FIG. 1 shows a general schematic diagram of an implementation of an X-ray facility,

FIG. 2 shows one configuration of the provision of the X-ray spectra in a first exemplary embodiment of a first approach,

FIG. 3 shows an illustration of an example resulting recording geometry,

FIGS. 4A,B schematically show an example representation of simultaneously occurring recording processes to explain the complete projection data recording,

FIG. 5 shows one possible multi-layered embodiment of the filtering facility in the first exemplary embodiment,

FIG. 6 shows a schematic view of a filtering structure in a second exemplary embodiment of the first approach,

FIG. 7 shows an example division of the X-ray detector resulting in the second exemplary embodiment,

FIG. 8 shows the configuration of the beam path in one exemplary embodiment of the second approach, and

FIG. 9 shows a flowchart of an exemplary embodiment of the method.

### DETAILED DESCRIPTION

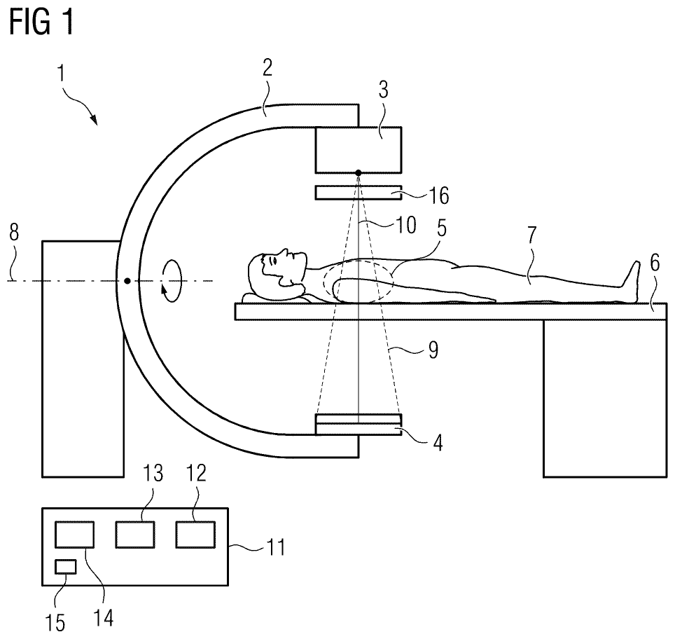

FIG. 1 shows a schematic diagram of an X-ray facility 1. This has a C-arm 2 on which an X-ray tube assembly 3 and an X-ray detector 4 are arranged opposite each other. The X-ray tube assembly 3 and the X-ray detector 4 form a recording arrangement. The C-arm 2 allows a movement of this recording arrangement at least in different rotatory degrees of freedom. In particular, a rotation of the recording arrangement about a recording region 5 of an examination object 7, here a patient, arranged on a patient couch 6 of the X-ray facility 1 can take place using a fixed axis of rotation 8 and a fixed plane of rotation (perpendicular to the image plane of FIG. 1 through a central beam 10) for covering an angle of projection range encompassing at least 360° (and also more).

An X-ray field 9 can be output to the X-ray detector 4 by the X-ray tube assembly 3 to record projection data of the recording region 5 from different directions of projection, which are defined by a central beam 10 of the X-ray field 9. In the present case, a cone beam geometry is used so a CBCT (cone beam CT) recording process therefore takes place.

Operation of the X-ray facility 1 is controlled by a control facility (system) 11, which is only indicated schematically. The control facility 11 has a recording unit (controller) 12 for controlling the recording operation and a reconstruction unit (processor or computer) 13, by which three-dimensional image datasets can be reconstructed from projection data of different directions of projection. Further, a control unit (controller) 14 for actuating further components of the X-ray facility 1 is provided. Various items of information, for example also recorded projection data, can be at least temporarily stored in a storage means (memory) 15.

In the present case, the X-ray facility 1 also includes a collimator and/or filter arrangement (filter or filter structure) 16, still generally shown here, which varies in its specific configuration according to embodiment and exemplary embodiment, as will be explained in more detail below. The specific exemplary embodiments discussed below relate to various options for simple and advantageous implementation of a dual-energy CBCT on the X-ray facility 1.

The X-ray field 9, and therefore also the X-ray detector, is divided into portions, which are permanently associated with the different X-ray spectra during the rotation. This means, X-ray radiation of one X-ray spectrum respectively is present in the portions of the X-ray field 9, with which spectrum the corresponding portion of the X-ray detector—after attenuation by the recording region 5—is exposed.

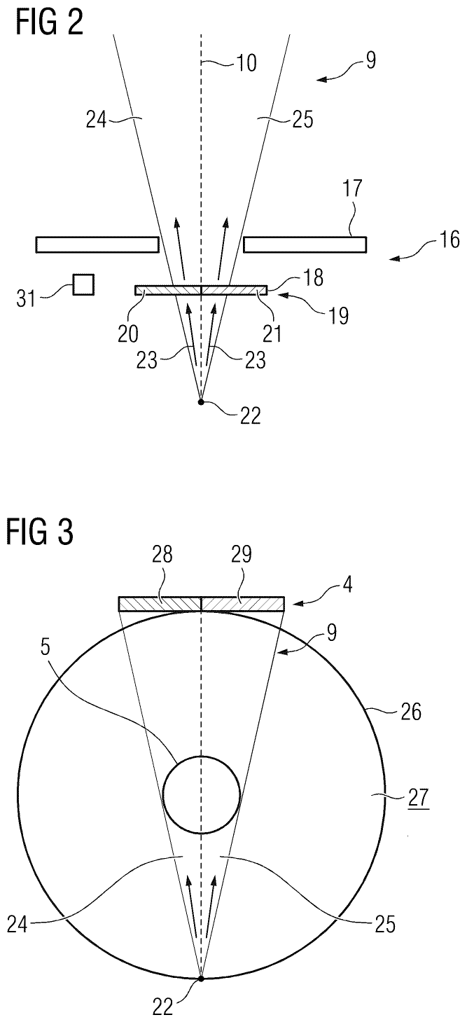

FIG. 2 shows a configuration of the provision of the X-ray spectra according to a first exemplary embodiment of a first approach. Accordingly, the collimator and/or filter arrangement 16, apart from a fundamentally known collimator 17 which predefines the dimensions of the X-ray field 7, includes a filtering facility 18 with a filtering structure 19, which in the present case has a first portion 20 and a second portion 21, which have different filtering properties. In this exemplary embodiment, the portions 20, 21 are embodied as halves since one side each of a central line of the filtering facility 16 passed through by the central beam 10 forms a portion 20, 21. The central line is embodied perpendicular to a plane of rotation (fixed during a dual-energy CBCT recording process), which corresponds in FIG. 2 to the image plane.

The X-ray tube assembly 3, starting from its (here exactly one) focus 22 provides an emission spectrum (indicated by the arrows 23), which is identical for the two portions, which spectrum strikes the filtering facility 18. The portions 20, 21 provide for a different kind of filtering, so after the filtering facility 18 the X-ray field 9 is divided into a first portion 24 of a first X-ray spectrum and a second portion 25 of a second X-ray spectrum. This division is fixed throughout the entire rotation, covering at least 360°, of the dual-energy CBCT recording process.

This will be explained once again in more detail by FIG. 3 where the recording trajectory 26, here a circular path, of the focus 22 during the rotation of the recording arrangement in the plane of rotation 27 is also shown. The filtering facility 18 is not shown for the sake of clarity, but the resulting division of the X-ray field 9 into the portions 24, 25 is. Since this division remains fixed, a fixed division into a portion 28 for recording projection data of the first X-ray spectrum and a portion 29 for recording projection data of the second X-ray spectrum is also provided on the detector surface of the X-ray detector 4, as shown in FIG. 3.

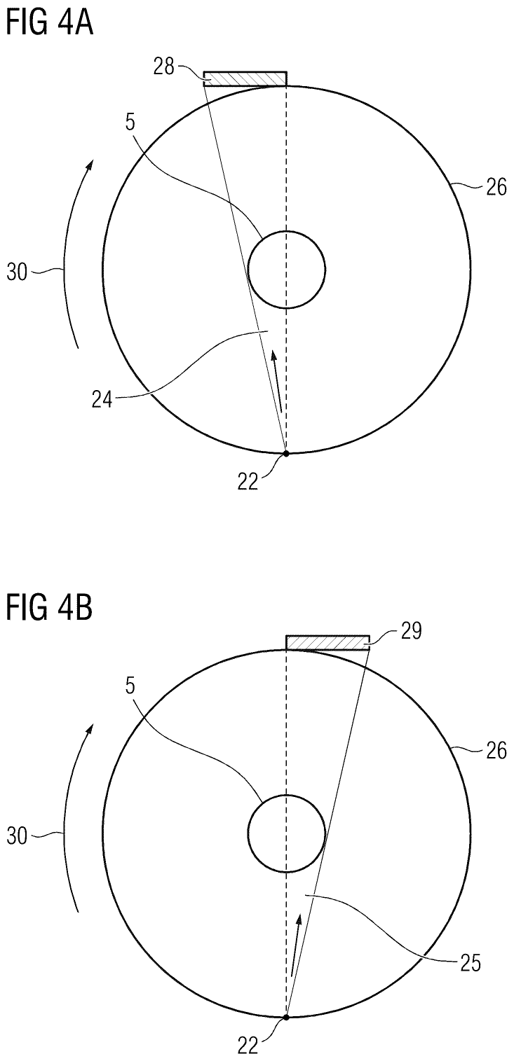

Since the recording takes place simultaneously, i.e., with the same X-ray pulse of the same focus 22, two partial recording processes therefore occur simultaneously. As shown in FIG. 4A and FIG. 4B: a complete 360° sampling, indicated by the arrow 30, of the recording region 5 with the first X-ray spectrum (portion 24 of the X-ray field 9) and the one detector half (portion 28), and a complete 360° sampling, indicated by the arrow 30, of the recording region 5 with the second X-ray spectrum (portion 25 of the X-ray field 9) and the other detector half (portion 29) are provided. It is known that the two partial recording processes enable a complete sampling with regard to the reconstruction of three-dimensional image datasets.

The filtering facility 18 can be introduced, for example as part of a filter wheel, into the beam path by an actuator 31 (indicated in FIG. 2) before the beginning of the dual-energy CBCT recording process, controlled by the control unit 14 of the control facility 11.

FIG. 5 shows one possible multi-layer construction of the filtering structure 19. This has a base layer 32, for example made of copper, which acts on the two portions 24, 25. Portion-specific layers 33 then provide the desired X-ray spectrum.

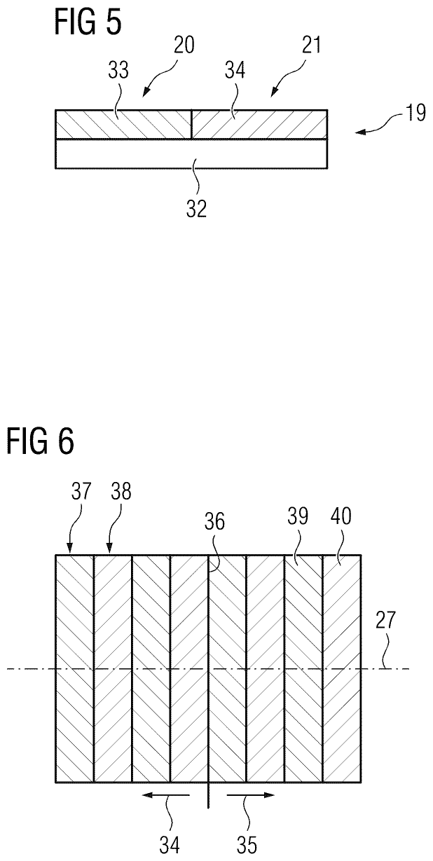

FIG. 6 shows as a second exemplary embodiment of the first approach a variant of the filtering structure 19, which can be advantageous with regard to movement correction, and to which the statements relating to the first exemplary embodiment continue to apply accordingly. It is not the entirety of the sides 34, 35, starting from the central line 36 of the filtering facility 18, which are consistently associated with the provision of an X-ray spectrum here, rather the portions 20, 21 include regions 37, 38, which in the present case are embodied as strips 39, 40 perpendicular to the plane of rotation 27. A type of comb structure therefore results. As already provided in the first exemplary embodiment, an antisymmetry to the central line 36 is provided. This means that for each point providing the first X-ray spectrum on the one side 34, 35 of the filtering structure 19 there exists, at the same distance from the central line on the other side 35, 34 of the filtering structure, a point providing the second X-ray spectrum.

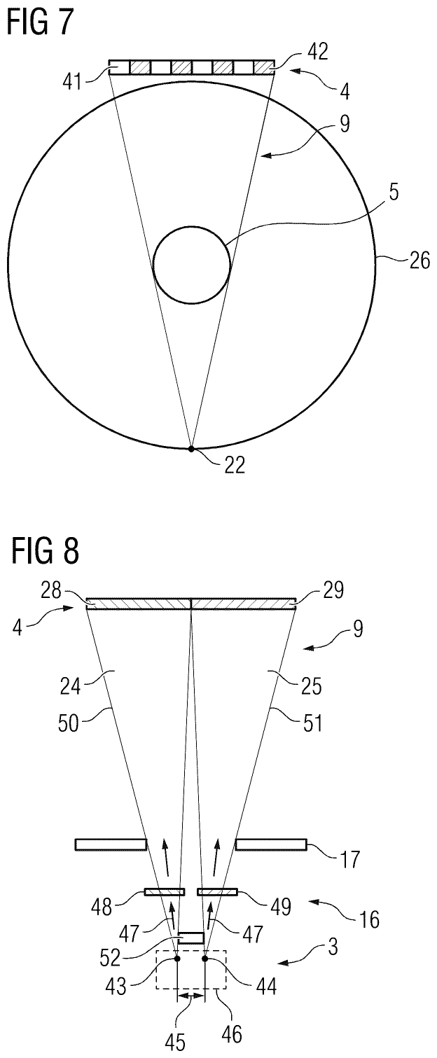

FIG. 7 shows the corresponding division of the detector surface of the X-ray detector 4 into corresponding strip-shaped regions 41, 42 of the portions 20, 21.

FIG. 8 shows an exemplary embodiment of the second approach. An X-ray tube assembly 3 is used here which has two X-ray focuses 43, 44, which in an X-ray tube 46 of the X-ray tube assembly 3 are outwardly offset by an identical small section respectively, for example 0.25 to 2.5 mm, from an imaginary central focal point of the central beam 10 (likewise imaginary here) of the X-ray field 9. The resulting focal distance 45 between the focuses 43, 44 can therefore be, for example, 0.5 to 5 mm. It is small compared to the focus-detector distance which can be, for example, 0.8 to 1.2 m.

The X-ray tube 46 of the X-ray tube assembly 3 can be actuated by the recording unit 12, to operate the focuses 43, 44 with different tube voltages, for example 70 and 140 kV. In addition, it is possible to quickly switch between the focuses 43, 44 and the tube voltages, for example with a switchover time of less than 1 ms. The X-ray focuses 43, 44 can therefore output different emission spectra (arrow 47) in a short interval, almost simultaneously therefore, from which spectra the different X-ray spectra are provided in the different portions 24, 25 of the X-ray field 9 by filters 48, 49. The emission spectra, and therewith the partial beam fields 50, 51, are separated by a central shading element 52. The width of the shading element is selected to be slightly smaller than the focal distance 45, so the partial beam fields 50, 51 of the X-ray field 9 at least substantially adjoin one another on the X-ray detector 4, again therefore one half each of the X-ray detector 4 can be used as the portion 28, 29 for measuring the projection data of the X-ray spectra, as represented in the upper region of FIG. 8. This therefore results in a recording process, as has already been explained with regard to FIGS. 3 and 4A, 4B. A change of filters 48, 49 is not necessary owing to the shading element 52.

Two specific configurations are conceivable with regard to the X-ray detector 4. It is thus firstly possible that the two portions, despite the successive exposure by the X-ray focuses 43, 44, are jointly readout in one readout cycle. It is also possible, however, at least for the first exposure process, to use a shadow register of the X-ray detector 4 to read out separately for the two X-ray spectra and exposures. In this case, the non-illuminated part respectively of the X-ray detector 4, including the portion 28, 29 of the other spectrum, is also read out in order to record scatter radiation data. This is used for a scatter radiation correction.

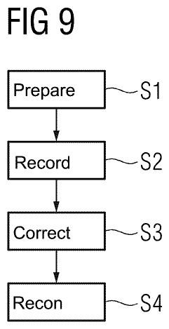

Finally, FIG. 9 shows a general flowchart for exemplary embodiments of the method.

If the filtering facility 18 or the filters 48, 49 have to be pivoted, the control unit 14 is used in an act S1 to introduce it/them into the beam path by the actuator 31. If the X-ray tube assembly 3 has to be put into a specific operating mode with regard to a plurality of focuses 43, 44 or the X-ray detector 4, for example for reading-out into a shadow register, then this can also occur by way of actuation by the control facility 11 in this preparatory act.

In an act S2, the recording arrangement is moved in a rotating manner, in particular using the recording trajectory 26, around the recording region 5 by the recording unit 12 and projection data is recorded for the two energy spectra in cone beam geometry projection from different directions of projection, here angles of projection. The rotation takes place to cover an angle of projection range incorporating at least 360°. The division of the X-ray field 9 and of the X-ray detector 4 always remains the same owing to the rotation of the entire recording arrangement (with the collimator and/or filtering arrangement 16); a coverage necessary for a complete reconstruction, as described, is nevertheless achieved. If there is a separate reading-out in the second approach and/or other shaded regions of the X-ray detector 4 are present due to the collimation by means of the collimator 17, then scatter radiation data is also recorded.

For the last-mentioned case of recording scatter radiation data, the scatter radiation data from the respectively shaded regions is used in an optional act S3 to ascertain a scatter radiation image for each set of projection data, which image is used for its correction with regard to the scatter radiation. This can take place, in particular, in a dedicated, correction unit (not shown here), or else as early as in the reconstruction unit 13 of the control facility 11.

Three-dimensional image datasets are reconstructed from the projection data in the reconstruction unit 13 in an act S4 for the two energy spectra. In the present case, the projection data of the respective other energy spectrum is also taken into account in the reconstruction of the three-dimensional image dataset of an energy spectrum, and, more precisely, in two ways in the present case: firstly for creating a truncation model, secondly for improved correction of cone beam artifacts. The two types can be based on iterative approaches in which firstly, first preliminary datasets are reconstructed from the respective sets of projection data, from which datasets it is possible to deduce items of information on extending the recording region 5, or patient 7 and/or items of material information. These can be used for estimating projection data missing due to truncation and/or for formulating boundary conditions for the subsequent reconstruction.

Although the invention has been illustrated and described in detail by the preferred exemplary embodiment, it is not limited by the disclosed examples and a person skilled in the art can derive other variations herefrom without departing from the scope of the invention.

Independent of the grammatical term usage, individuals with male, female or other gender identities are included within the term.

1. A method for dual-energy imaging of a recording region with an X-ray system, which has a recording arrangement with an X-ray tube assembly and an X-ray detector for receiving X-rays of an X-ray field emitted by the X-ray tube assembly in cone beam geometry, which field has a central beam, the method comprising:
rotating the recording arrangement around the recording region;
recording projection data of different directions of projection during the rotating, the projection data recorded for two different X-ray spectra, wherein the projection data of the two X-ray spectra is recorded during the rotation that covers at least 360°; and
reconstructing a three-dimensional image dataset of the recording region for each X-ray spectrum from the respective projection data;
wherein, for each X-ray spectrum, an associated portion of the X-ray field is fixed over the rotation, and a corresponding, associated, fixed portion of the X-ray detector is used for recording.

2. The method as claimed in claim 1, wherein, along a central line running perpendicular to a rotational plane through the incidence of the central beam the X-ray detector is divided into two sides, wherein the X-ray spectra are divided into the fixed portions antisymmetrically in respect of the central line.

3. The method as claimed in claim 1, wherein a filtering system arranged between the X-ray tube assembly and the recording region is used for the definition of the associated and fixed portions, which filtering system has a filtering structure penetrated by the X-ray field, with a first portion for providing a first X-ray spectrum of the X-ray spectra and a second portion for providing a second X-ray spectrum of the X-ray spectra.

4. The method as claimed in claim 3, wherein the first and second portions of the filtering structure comprise a plurality of regions, respectively, which are separated from each other by regions of the other of the first and second portions.

5. The method as claimed in claim 4, wherein the regions are strips running perpendicular to a rotational plane.

6. The method as claimed in claim 1, wherein the X-ray tube assembly has two focuses associated with the different X-ray spectra, the two focuses physically spaced apart by a focal distance, which focuses follow one another in a rotational plane, wherein the focuses are operated with different tube voltages, and partial beam fields emitted by the focuses are separated by a shading element arranged in a beam path between the focuses.

7. The method as claimed in claim 6, wherein the focal distance is 0.5 to 5 mm and/or the width of the shading element is less than the focal distance.

8. The method as claimed in claim 6, wherein the focuses are generated in a same X-ray tube of the X-ray tube assembly, wherein the different tube voltages are switched sequentially.

9. The method as claimed in claim 8, wherein the portions of the X-ray detector sequentially illuminated by the X-ray spectra are read out in a joint readout cycle.

10. The method as claimed in claim 8, wherein the portions of the X-ray detector sequentially illuminated by the partial beam fields are successively read out.

11. The method as claimed in claim 10, wherein, with the reading out of each portion, the part of the X-ray detector not covered by the respective partial beam field, comprising the portion of the other partial beam field, is also read out, obtaining scatter radiation data used in a scatter radiation correction.

12. The method as claimed in claim 1, wherein, for the reconstruction of the image dataset for one of the X-ray spectra respectively, projection data of the other X-ray spectrum is taken into account.

13. The method as claimed in claim 12, wherein a first reconstruction of preliminary datasets takes place from the respective projection image data, an item of material information describing a distribution of material in respect of the recording region is ascertained from the preliminary image datasets and the item of material information is taken into account by at least one boundary condition and/or in a target function during at least one new reconstruction from the respective projection data.

14. The method as claimed in claim 12, wherein the projection data of the two X-ray spectra is used for ascertaining a truncation model taken into account during the reconstruction of the two image datasets.

15. An X-ray system comprising:
a recording arrangement with an X-ray tube assembly and an X-ray detector for receiving X-rays of an X-ray field emitted by the X-ray tube assembly in cone beam geometry, which has a central beam; and
a controller configured to
rotate the recording arrangement around a recording region;
record projection data of different directions of projection during the rotation, the projection data recorded for two different X-ray spectra, wherein the projection data of the two X-ray spectra is recorded during the rotation that covers at least 360°; and
reconstruct a three-dimensional image dataset of the recording region for each X-ray spectrum from the respective projection data;
wherein, for each X-ray spectrum, an associated portion of the X-ray field is fixed over the rotation, and a corresponding, associated, fixed portion of the X-ray detector is used to record the projection data.

16. The X-ray system as claimed in claim 15, wherein, along a central line running perpendicular to a rotational plane through an incidence of the central beam, the X-ray detector is divided into two sides, wherein the X-ray spectra are divided into the fixed portions antisymmetrically in respect of the central line.

17. The system as claimed in claim 15, further comprising a filtering system arranged between the X-ray tube assembly and the recording region, the filtering system defining the fixed portions, which filtering system has a filtering structure penetrated by the X-ray field, with a first portion for providing a first X-ray spectrum of the X-ray spectra and a second portion for providing a second X-ray spectrum of the X-ray spectra.

18. The system as claimed in claim 15, wherein the X-ray tube assembly has two focuses associated with the different X-ray spectra, the two focuses physically spaced apart by a focal distance, which focuses follow one another in a rotational plane, wherein the focuses are operated with different tube voltages, and partial beam fields emitted by the focuses are separated by a shading element arranged in a beam path between the focuses.
